(19) United States
(12) Patent Application Publication
(10) Pub. No.: US 2024/0108593 A1
(43) Pub. Date: Apr. 4, 2024

(54) METHOD OF TREATING POLYAMINE IMBALANCE-RELATED DISORDERS

(71) Applicant: UNIVERSITY OF MIAMI, Miami, FL (US)

(72) Inventors: Rong Grace Zhai, Miami, FL (US); Xianzun Tao, Miami, FL (US); Chong Li, Miami, FL (US); Yi Zhu, Miami, FL (US); Zoraida Diaz-Perez, Miami, FL (US)

(73) Assignee: UNIVERSITY OF MIAMI, Miami, FL (US)

(21) Appl. No.: 18/039,897

(22) PCT Filed: Dec. 6, 2021
(86) PCT No.: PCT/US21/61973

§ 371 Date: Jun. 1, 2023

Related U.S. Application Data
(60) Provisional application No. 63/122,251, filed on Dec. 7, 2020.

(51) Int. Cl. A61K 31/192; A61P 43/00; C12Q 1/6883

(52) U.S. Cl. CPC A61K 31/192; A61P 43/00; C12Q 1/6883; C12Q 2600/156

(57) ABSTRACT

The disclosure provides a method of treating a polyamine imbalance-related disorder. The method comprises administering phenylbutyrate to a subject in need thereof, thereby treating the polyamine imbalance-related disorder.

### CROSS REFERENCE TO RELATED APPLICATION

This application claims the benefit of priority to U.S. Provisional Patent Application No. 63/122,251, filed on Dec. 7, 2020, the disclosure of which is hereby incorporated by reference in its entirety.

### GOVERNMENT SUPPORT CLAUSE

This invention was made with government support under grant number 1R01NS109640, awarded by the National Institutes of Health. The government has certain rights in the invention.

### FIELD OF DISCLOSURE

The disclosure relates to methods for treating polyamine imbalance-related disorders, such as Snyder-Robinson syndrome.

### BACKGROUND

Polyamines are an essential class of metabolites that play a role in gene expression, cell growth and proliferation, and stress response. Common polyamines include spermidine, spermine, putrescine, and cadaverine. These polyamines are ubiquitously present in many organisms, and dysregulation of polyamines has been observed in connection with several diseases.

Snyder-Robinson Syndrome (SRS) is a rare disease directly linked to a genetic defect in the polyamine biosynthesis pathway. The causative X-linked gene for SRS was first identified when a splice mutation was found in the spermine synthase gene (SMS) resulting in truncation of the protein. Cason et al., Eur J Hum Genet, 2003. 11(12): p. 937-44. Since then, thirteen additional missense mutations have been reported. See, e.g., Li et al., Nature Comm, 2017. 8:1257; Albert et al., Orphanet J Rare Dis, 2015. 10: p. 27; Becerra-Solano et al., Am J Med Genet A, 2009. 149A(3): p. 328-35; de Alencastro et al., J Med Genet, 2008. 45(8): p. 539-43; and Zhang et al., Hum Mol Genet, 2013. 22(18): p. 3789-97. Spermine synthase (SMS) catalyzes the conversion of spermidine to spermine. Pegg and Michael, Cell Mol Life Sci, 2010. 67(1): p. 113-21. All identified SMS mutations cause a partial to complete loss of the enzymatic activity. The enzymes in polyamine metabolism have been underappreciated until the discovery of the casual link between mutations in spermine synthase (SMS) and SRS. Indeed, although the biochemical properties of SMS are well characterized, the nervous system-specific function of SMS, and polyamine metabolism in general, are largely unknown.

### SUMMARY

The disclosure provides a method of treating a polyamine imbalance-related disorder. The method comprises administering phenylbutyrate to a subject in need thereof, thereby treating the polyamine imbalance-related disorder. In various aspects of the disclosure, the polyamine imbalance-related disorder is a genetic disorder, such as Snyder-Robinson syndrome or Bachmann-Bupp syndrome.

Additional features and variations of the invention will be apparent to those skilled in the art from the entirety of this application, including the figures and detailed description, and all such features are intended as aspects of the invention. Likewise, features of the invention described herein can be re-combined into additional embodiments that also are intended as aspects of the invention, irrespective of whether the combination of features is specified as an aspect or embodiment of the invention. The entire document is intended to be related as a unified disclosure, and it should be understood that all combinations of features described herein (even if described in separate sections) are contemplated, even if the combination of features is not found together in the same sentence, or paragraph, or section of this document. Also, only such limitations which are described herein as critical to the invention should be viewed as such; variations of the invention lacking limitations which have not been described herein as critical are intended as aspects of the invention.

### BRIEF DESCRIPTION OF THE DRAWINGS

FIG. 1 is an illustration of cellular responses to altered polyamine catabolism caused by SMS deficiency. SMS is an aminopropyltransferase for catalyzing spermidine into spermine. SMS deficiency leads to spermidine buildup and catabolism. The accumulated catabolic metabolites, H₂O₂ and aldehydes, cause oxidative stress and lysosomal dysfunction, which further impair autophagy flux, endocytosis and mitochondria function.

FIG. 2 is a graph summarizing the results described in the Examples which demonstrated that sodium phenylbutyrate (PBA) extends lifespan of SMS mutant flies. SMS mutant flies (1-3 days after eclosion (DAE)) were raised in vials with 10 mL of gel-solid cornmeal-based food with indicated concentration of inhibitor (dissolved in water). Around 10-20 flies were placed in each vial and food was changed every week. N=76/77/53/53 for No PBA treatment (squares)/2 mM (circles)/5 mM (triangles)/10 mM (upside-down triangles) groups, respectively. ****p<0.001, Statistic method: Log-rank (Mantel-Cox) test.

FIGS. 3A and 3B demonstrate that phenylbutyrate (here, PBA) reduces ROS level and rescues brain size of SMS mutant flies. Control and SMS mutant flies were fed with food containing 0 or 2 mM of PBA for 10 days. Brains were dissected and immediately live-stained for DHE. Brain size and DHE signal intensity (indicating ROS level) were quantified and analyzed in ImageJ. Mean ROS level was calculated by dividing DHE signal intensity by brain size. animals for each group. Data, mean±S.E.M., *P<0.05, ** P<0.01, ***P<0.005. Unpaired t test (two-tailed).

FIG. 4 demonstrates that phenylbutyrate (here, PBA) reduces aldehyde level of SMS mutant flies. Control (x axis—+/+) and SMS mutant (x axis—−/−) flies were fed with food containing 0 (PBA−) or 2 mM (PBA+) of PBA for 10 days. Flies were homogenized and the aldehyde level was measured according to the instruction from the kit manufacturer (Sigma, cat #MAK141). The y-axis denotes aldehyde levels (nmol/mg of tissue). n≥3 animals for each group. Data, mean±S.E.M., ** P<0.01, Student's t test.

FIG. 5 demonstrates that phenylbutyrate (here, PBA) improves mitochondrial COX activity of SMS mutant flies. Control (x axis—+/+) and SMS mutant (x axis—−/−) flies were fed with food containing 0 (PBA−) or 2 mM (PBA+) of PBA for 10 days. Fly flight muscle was dissected and stained for 3,3′-diaminobenzidine (DAB). DAB signal intensity (indicating mitochondrial COX activity) was quantified and analyzed in ImageJ. n≥3 animals for each group. Data, mean±S.E.M., ***P<0.001, Student's t test.

FIG. 6 demonstrates that phenylbutyrate (here, PBA) improves autophagy flux of SMS mutant flies. Control (x axis—+/+) and SMS mutant (x axis—−/−) flies were fed with food containing 0 (PBA−) or 2 mM (PBA+) of PBA for 10 days. Fly heads were homogenized and probed for Ref(2)p and Actin by western blot. Ref(2)p and Actin bands were quantified by ImageJ. Ref(2)p reduction indicates improved autophagy flux. n≥3 samples for each group. Data, mean±S.E.M., *P<0.05, ***P<0.001, Student's t test.

FIGS. 7A and 7B demonstrate that phenylbutyrate (here, PBA) improves lysosomal cathepsin maturation and autophagy flux in SRS patient fibroblast cells. SRS patient Q148R/I150T fibroblasts were treated with at 0, 0.5 or 2.0 mM PBA for 48 hours. Patient and age-matched control cells were lysed and probed for cathepsin, p62 and Actin by western blot. p62 reduction indicates improved autophagy flux. Mature and pro-Cts D, p62 and Actin bands were quantified by ImageJ. n≥3 samples for each group. Data, mean±S.E.M., *P<0.05, Student's t test.

### DETAILED DESCRIPTION

The disclosure provides a method of treating a polyamine imbalance-related disorder. The method comprises administering phenylbutyrate to a subject in need thereof, thereby treating the polyamine imbalance-related disorder. Polyamine imbalance-related disorders are disorders associated with dysregulation of polyamine metabolism, which results in increased or decreased levels of, e.g., spermidine, spermine, putrescine, and cadaverine. In various aspects, the polyamine-imbalance disorder is associated with aberrantly increased levels of spermidine. Examples of polyamine-imbalance disorders include neurodegenerative diseases, such as Parkinson's disease and Alzheimer's disease. Parkinson's patients have higher concentrations of putrescine, cadaverine, N1-acetyl-cadaverine, and N1-acetyl-spermidine, as well as lower levels of spermidine in the cerebrospinal fluid. Alzheimer's patients also demonstrate polyamine imbalance, wherein putrescine, spermine, and spermidine levels are increased. See, e.g., Miller-Fleming et al., Journal of Molecular Biology, 2015. 427(21): p. 3389-3406; Roede et al., PLoS One, 2013. 8; p. e77629; and Inoue et al., Sci. Rep., 2013, 3: p. 2364. Polyamine imbalance-related disorders are those that positively respond to restoration of polyamine metabolism.

In various aspects of the disclosure, the polyamine imbalance-related disorder is a genetic disorder. Examples of genetic disorders associated with polyamine imbalance include, e.g., Snyder-Robinson syndrome (SRS) and Bachmann-Bupp syndrome (BABS). Snyder-Robinson syndrome (SRS) is directly linked to a genetic defect in the polyamine biosynthesis pathway (i.e., one or more mutations in a gene encoding a protein in the polyamine biosynthesis pathway). SRS is a rare, X-linked intellectual disability disorder caused by mutations in the Spm synthase gene (c.831G>T:p.L277F). These mutations reduce the levels of spermine (Spm), leading to an imbalance in spermidine/spermine (Spd/Spm) ratios. This imbalance leads to intellectual disability, as well as other possible symptoms such as enlarged brain volume, osteoporosis, broad-based gait, facial dysmorphism, seizures, frequent bone fractures, reduced muscle tone, speech abnormalities, and curvature of the spine.

Bachmann-Bupp syndrome is caused by mutations in the ornithine decarboxylase 1 (ODC1) gene, and is also associated with intellectual disability or delay alongside a number of other potential clinical features including macrosomia, macrocephaly, alopecia, spasticity, hypotonia, cutaneous vascular malformation, delayed visual maturation, and sensorineural hearing loss. The ODC1 gene mutation (c.1342 A>T) was originally identified by whole exome sequencing and confirmed by Sanger sequencing.

The disclosure provides a method of treating a polyamine imbalance-related disorder. “Treatment” does not require complete remission or cure of the disorder; any improvement in the disorder and/or improvement in the symptoms associated with the disorder are contemplated. The method may also, in various aspects, slow the progression of a disorder, which is considered herein as “treatment.” For example, in the context of neurological diseases such as SRS, a therapeutic response would refer to improvements in one or more clinical manifestations of the disorder, such as enhanced neurological function; enhanced neuromuscular function; improvement in speech, hearing, or vision; achievement of developmental milestones; weight, and improvement in muscle mass or tone. SRS disease state is monitored by, e.g., clinical examination, computerized tomography (CT), magnetic resonance imaging (MRI; e.g., functional MRI to evaluate brain activity), and intellectual development testing. Thus, in various aspects, the method of the disclosure improves neurological or intellectual impairment associated with a polyamine imbalance-related disorder, such as SRS. Treatment also may be characterized by restoration, at least in part, of normal polyamine levels in the subject. Thus, in various aspects, the disclosure provides a method of reducing spermidine levels in a subject in need thereof, the method comprising administering phenylbutyrate to the subject.

The “subject” of the method is a mammalian subject, e.g., a human. In various aspects, the subject is a male. In various aspects, the subject is aged 18 years or less (e.g., a child aged 11 years or less or an adolescent aged 12-18 years).

Optionally, the method comprises determining whether a subject is suffering from polyamine imbalance. In this regard, the method may comprise obtaining a biological sample from a subject and determining the presence of a mutation in one or more genes associated with polyamine metabolism, determining aberrant enzyme activity in a polyamine metabolic pathway, and/or determining aberrant levels of one or more polyamines (e.g., putrescine, spermidine, spermine, or acetylated versions thereof) in the sample. The biological sample may be any biological sample comprising the subject's genetic material (DNA or RNA) for sequencing or quantification. Alternatively or in addition, the biological sample is a sample suitable for identifying and/or measuring endogenous polyamine levels or evaluating enzyme activity. In various aspects, the biological sample is blood, cerebrospinal fluid, or urine.

In various aspects, the method comprises detecting mutations in one or more genes encoding proteins associated with polyamine metabolism, e.g., ODC, DHPS, DOHH, AMD1, MAT1A, MAT1B, SRM, SMS, SMOX, MYC, SAT1, PAOX, or EIF5A. Optionally, the method comprises detecting one or more mutations in the SMS gene. DNA sequencing methods are well known in the art and include but are not limited to, Sanger sequencing, massively parallel signature sequencing (MPSS), single molecule real time (SMRT) sequencing, Illumina (Solexa) sequencing, nanopore DNA sequencing, and other next generation sequencing methods. In various aspects, the method comprises characterizing the activity of a peptide and/or enzyme in the polyamine metabolic pathway. Methods of determining the activity of an enzyme are known in the art. In various aspects, the method comprises measuring the levels of a polyamine in the sample. Any method suitable for measuring the level of polyamine (e.g., putrescine, spermidine, spermine, or acetylated versions thereof) in a biological sample may be used. Exemplary methods include, but are not limited to, reverse-phase HPLC and mass spectrometry. Aberrant levels of enzyme activity or polyamine are identified by comparing the measurements obtained in the biological sample to a control sample (e.g., a sample from a healthy subject or subject who is not afflicted with a polyamine imbalance-related disorder). In various aspects of the disclosure, the method comprises identifying a subject having a mutation in the Spm synthase gene. In various aspects, the mutation is selected from SMS mutations c. 104T>A*/G, c. 166G>T*/A, c.174T>A, c.200G>A, c.329+5G>A, c.335C>T, c.395G>T, c.443A>G, c.449T>C, c.638C>A*, c.699G>A*, c.905 C>T, c.908_911del, c.983A>G, or c.831 G>T:p.L277F. In various aspects, the method comprises identify a subject having a mutation in the ornithine decarboxylase gene. 1 In various aspects, the mutation is ODC1 mutation c.1342 A>T.

The method described herein comprises administering phenylbutyrate to a subject in need thereof. By “phenylbutyrate” is meant phenylbutyrate or a prodrug or salt thereof. “Prodrug” refers to a moiety that releases a compound when administered to a patient. Prodrugs can be prepared by modifying functional groups present in the compounds in such a way that the modifications are cleaved, either in routine manipulation or in vivo, to the parent compound. A phenylbutyrate salt refers to a salt that retains the biological effectiveness and properties of phenylbutyrate and which is suitable for pharmaceutical use. Generally, pharmaceutically acceptable acid addition salts can be formed with inorganic acids and organic acids. Examples of inorganic acids from which salts can be derived include, but are not limited to, hydrochloric acid, hydrobromic acid, sulfuric acid, nitric acid, and phosphoric acid. Organic acids from which salts can be derived include, but are not limited to, acetic acid, benzoic acid, cinnamic acid, citric acid, fumaric acid, glycolic acid, maleic acid, malonic acid, propionic acid, pyruvic acid, oxalic acid, succinic acid, tartaric acid, mandelic acid, methanesulfonic acid, ethanesulfonic acid, p-toluenesulfonic acid, and salicylic acid. Pharmaceutically acceptable salts can also be formed using inorganic bases (e.g., bases that contain sodium, potassium, lithium, ammonium, calcium, magnesium, iron, zinc, copper, manganese, aluminum) or organic bases. In various aspects, “phenylbutyrate salt” refers to a complex containing phenylbutyrate as the anion and, e.g., any biologically suitable cation having a +1 charge. Suitable cations include those in group 1 of the periodic table, such as Li+, Na+, K+. Other examples of suitable cations include metals having a +1 charge, such as Cu+ and Ag+, and polyatomic ions having a +1 charge (e.g., NH₄+). In various aspects, the method comprises administering sodium phenylbutyrate (C₁₀H₁₁NaO₂; 4-phenylbutyric acid, sodium salt) to the subject. Glycerol phenylbutyrate also is contemplated. Sodium phenylbutyrate is commercially available as BUPHENYL®. Glycerol phenylbutyrate is commercially available as RAVICTI™.

The phenylbutyrate or prodrug or salt thereof may be administered to a subject in any pharmaceutically acceptable formulation using any route of administration appropriate for achieving the desired biological effect. Methods of administration may include, but are not limited to, oral administration and parenteral administration, including, but not limited to, intradermal, intramuscular, intraperitoneal, intravenous, intraarterial, subcutaneous, epidural, sublingual, intranasal, intracerebral, intraventricular, intrathecal, intravaginal, transdermal, rectally, inhalation, intrapulmonary, intra-airway, intrabronchial, intratracheal, or topical (e.g., to the ears, nose, eyes, or skin) delivery.

Formulations containing the phenylbutyrate or prodrug or salt thereof and a suitable carrier can be provided in solid dosage forms including, but not limited to, softgels, tablets, capsules, cachets, pellets, pills, powders, and granules; topical dosage forms including, but not limited to, solutions, powders, fluid emulsions, fluid suspensions, semi-solids, ointments, pastes, creams, gels and jellies, and foams; and parenteral dosage forms including, but not limited to, solutions, suspensions, emulsions, and powders. The phenylbutyrate or prodrug or salt thereof may be contained in formulations with pharmaceutically acceptable diluents, fillers, disintegrants, binders, lubricants, surfactants, hydrophobic vehicles, water-soluble vehicles, emulsifiers, buffers, humectants, moisturizers, solubilizers, preservatives, and the like. The means and methods for administration are known in the art and an artisan can refer to various pharmacologic references for guidance. See, for example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980). In exemplary aspects of the disclosure, the phenylbutyrate or prodrug or salt thereof is administered via ingestion (e.g., via mouth, gastrostomy, or nasogastric tube) and is provided in the form of a tablet, powder, or solution for ingestion.

The method preferably comprises administering an effective amount of the phenylbutyrate or prodrug or salt thereof to the subject. The term “effective amount” as used herein refers to an amount of a therapeutic agent needed to treat or ameliorate a targeted condition, or to exhibit a detectable beneficial biological effect over a clinically relevant time period. The dose of a pharmaceutical composition administered to a subject may vary depending on various factors, including the patient's age, weight, general health condition, sex, diet, the duration of administration, the route of administration, and the severity of the particular disorder to be treated. A pharmaceutical composition may be administered once or several times a day, or individual doses may be administered at longer intervals (e.g., once every week, once a month, etc.). In various aspects, the subject is an infant or child weighing less than 20 kg, and 450-600 mg/kg/day of phenylbutyrate or prodrug or salt thereof is administered orally. In various aspects, the subject is a child weighing 20 kg or more or an adolescent, and 9.9-13 g/m²/day of phenylbutyrate or prodrug or salt thereof is administered orally. Optionally, a daily dose is provided in divided doses (i.e., multiple administrations) three to six times throughout the day.

The disclosure also provides use of phenylbutyrate in the treatment of a polyamine imbalance-related disorder in a subject in need thereof, as well as phenylbutyrate for use in the in the treatment of a polyamine imbalance-related disorder in a subject in need thereof. In various aspects, the polyamine imbalance-related disorder is a genetic disorder, such as Snyder-Robinson syndrome or Bachmann-Bupp syndrome. Optionally, the subject is 18 years old or younger. In some aspects of the disclosure, the phenylbutyrate is administered as sodium phenylbutyrate. Also, the method optionally further comprises, prior to the administration step, detecting a genetic defect in the polyamine biosynthesis pathway in a biological sample obtained from the subject. For example, the genetic defect may be a mutation in the spermine (Spm) synthase gene or a mutation in the ornithine decarboxylase 1 (ODC1) gene.

### Example

This Example describes the beneficial impact of phenylbutyrate in a model of polyamine-imbalance disorders (SRS) and in SRS patient samples.

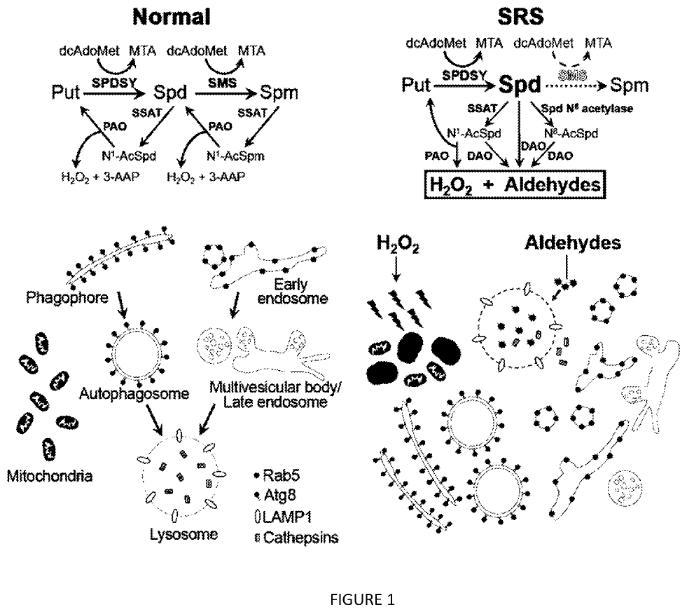

We established a Drosophila model of SRS and found that human and Drosophila SMS proteins are functionally conserved, and loss of SMS in Drosophila recapitulated the pathological polyamine imbalance of SRS and caused reduced survival rate and synaptic dysfunction. Imaging and ultrastructural analyses of Drosophila brain detected abnormal mitochondrial morphology and lysosome damage in SMS mutant neurons, suggesting mitochondrial and lysosomal toxicity as detrimental consequences of abnormal polyamine catabolism. As H₂O₂ and amino aldehydes are two byproducts of polyamine catabolism, reactive oxygen species (ROS) and aldehyde levels were analyzed; elevated levels of both neurotoxic molecules were detected in SMS mutant flies, supporting a model of SRS pathology, where H₂O₂ and amino aldehydes cause distinct cellular damage that, in combination, result in neuronal damage (FIG. 1). Genetic and pharmacological suppressors of ROS were tested, resulting in only a modest reduction of mitochondrial toxicity in SMS mutant flies. However, suppressors of ROS (antioxidants) showed no effect on lysosome damage recovery, which explains inconsistent responses observed in patients administered other therapeutics and highlights the need for better treatment options for SRS.

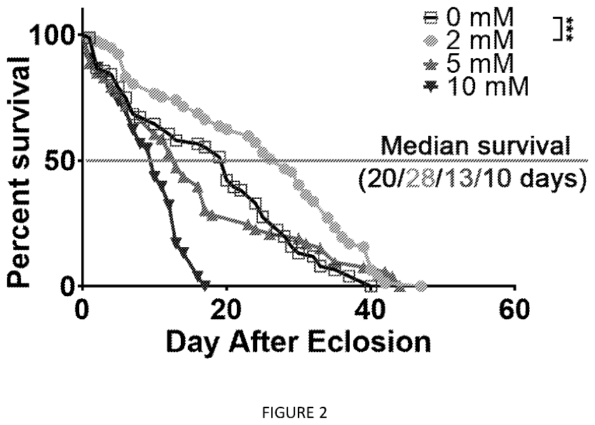

In a campaign to identify alternative treatment options for SRS, sodium phenylbutyrate (PBA) was surprisingly determined to have a protective effect in a Drosophila model of SRS and in SRS patient cells. PBA was tested in feeding experiments. SMS homozygous mutant flies 1-3 days old (DAE, days after eclosion) were fed with PBA-containing food. Survival rate was scored every day, and lifespan curve was plotted. As shown in FIG. 2, feeding with 2 mM PBA significantly extended the lifespan of SMS mutant flies. The mid-point 50% survival increased from 19 days to 27 days, representing a 42% increase in survival.

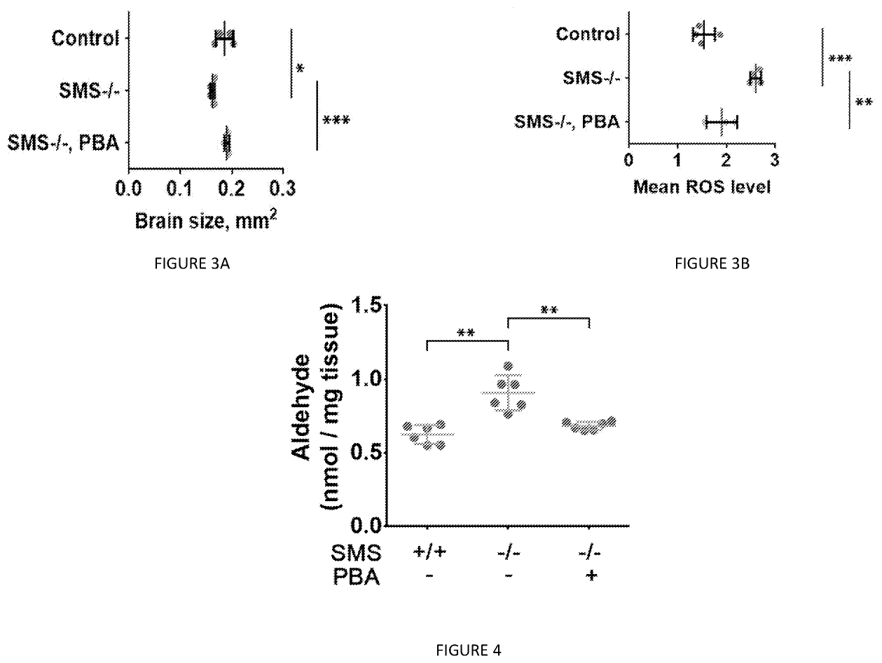

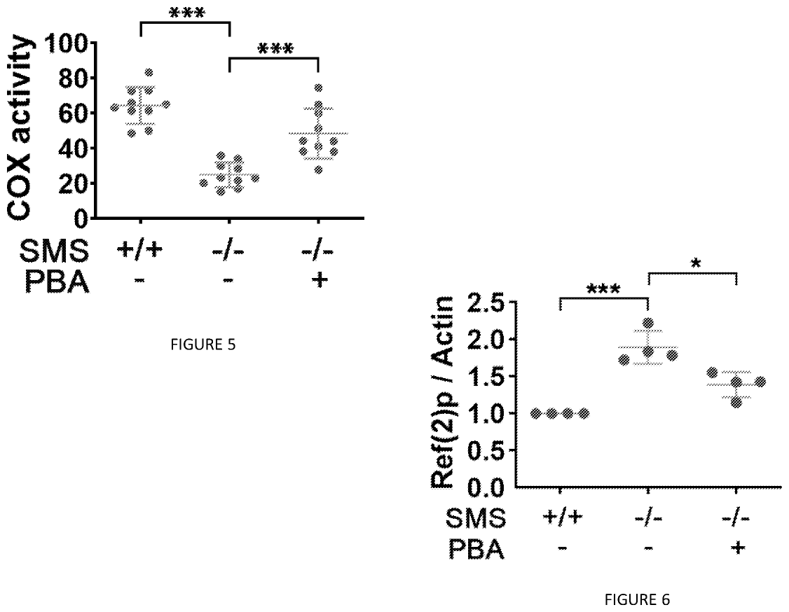

The effect of PBA on morphological phenotypes in the mutant brain was examined. Loss-of-SMS resulted in significant elevation of brain ROS levels, detected by dihydroethidium (DHE) staining. As shown in FIG. 3B, compared to control brains, SMS mutant brains have significantly higher level of ROS. In addition, SMS mutant flies have slightly reduced brain size. See FIG. 3A. Strikingly, SMS mutant flies fed with 2 mM PBA for 10 days not only showed significantly reduced level of ROS in the brains, but also have normal size, indistinguishable from controls, suggesting much improved brain health. The brain morphological analyses further indicated that PBA may improve nervous system function and the overall health of mutant animals. The data illustrated in FIGS. 4-6 further demonstrate that PBA improves the health of SRS cells and tissue.

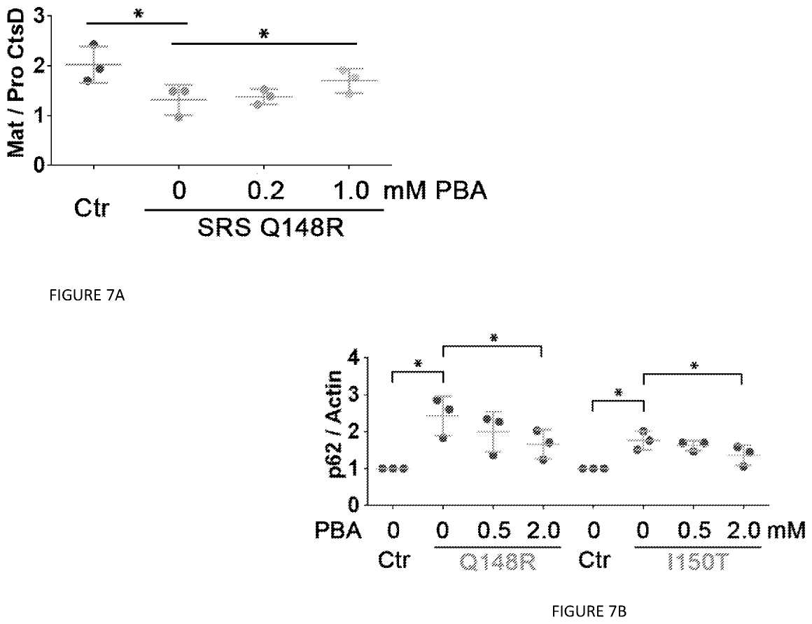

The exciting beneficial effects observed in the SRS fly models prompted confirmation of the effect of PBA in human patient samples. Assays were conducted using fibroblasts from SRS patients carrying a Q148R mutation. Q148R patient fibroblasts have lysosomal dysfunction, as evidenced by incomplete processing of the lysosomal resident enzyme cathepsin due to lysosomal acidification defect. Compared to age-matched control fibroblasts, the ratio of mature to pro-cathepsin D (CtsD) was significantly lower in SRS Q148R fibroblasts, and the level of partially processed, intermediate CtsD (Int CtsD) was higher. These results indicate incomplete cathepsin maturation in damaged lysosomes in SRS fibroblasts. Treating SRS fibroblasts with the PBA significantly improved the ratio of mature to pro CtsD at the concentration of 1.0 mM (FIGS. 7A and 7B), indicating the partial recovery of lysosomal function. These results in SRS patient fibroblasts were consistent with the in vivo observations in Drosophila model, confirming the conservation of pathology and underlying mechanisms.

Collectively, the data in Drosophila and SRS patient cells indicate that PBA reduced ROS accumulation, rescued lysosome integrity, enhanced neuronal survival, and extended lifespan in vivo in Drosophila and in SRS patient cells. The data showing that PBA treatment partially rescues lysosomal Cathepsin D maturation in SRS patient fibroblasts indicates the recovery of damaged lysosomes and the beneficial effects for SRS patients. In addition to SRS, the data suggest that PBA treatment would benefit other polyamine imbalance-related disorders, including Bachmann-Bupp Syndrome (BABS), caused by mutations of another polyamine pathway enzyme, ornithine decarboxylase 1 (ODC1). See, e.g., Schultz et al., Biochem J, 2019. 476(14): p. 2047-2057. In these patients, mutant ODC1 enzyme is more stable and accumulated, which results in significantly increased intracellular putrescine and spermidine. Restoration of normal polyamine metabolism would provide a beneficial effect in the disease state of such patients.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context; the terms “a” (or “an”), “one or more,” and “at least one” can be used interchangeably herein. The term “or” should be understood to encompass items in the alternative or together, unless context unambiguously requires otherwise. The term “and/or” should be understood to encompass each item in a list (individually), any combination of items a list, and all items in a list together. The terms “comprising,” “having,” “including,” and “containing” are to be construed as open-ended terms (i.e., meaning “including, but not limited to,”) unless otherwise noted. It should be understood that, while various embodiments in the specification are presented using “comprising” language, under various circumstances, a related embodiment may also be described using “consisting of” or “consisting essentially of” language. The disclosure contemplates embodiments described as “comprising” a feature to include embodiments which “consist of” or “consist essentially of” the feature.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

What is claimed is:

1. A method of treating a polyamine imbalance-related disorder, the method comprising administering phenylbutyrate to a subject in need thereof, thereby treating the polyamine imbalance-related disorder.

2. The method of claim 1, wherein the polyamine imbalance-related disorder is a genetic disorder.

3. The method of claim 2, wherein the disorder is Snyder-Robinson syndrome.

4. The method of claim 2, wherein the disorder is Bachmann-Bupp syndrome.

5. The method of any one of claims 1-4, wherein the subject is 18 years old or younger.

6. The method of any one of claims 1-5, wherein the phenylbutyrate is administered as sodium phenylbutyrate.

7. The method of any one of claims 1-6, wherein the method further comprises, prior to the administration step, detecting a genetic defect in the polyamine biosynthesis pathway in a biological sample obtained from the subject.

8. The method of claim 7, wherein the genetic defect is a mutation in the spermine (Spm) synthase gene.

9. The method of claim 7, wherein the genetic defect is a mutation in the ornithine decarboxylase 1 (ODC1) gene.
